(19) United States
(12) Plant Patent Application Publication
(10) Pub. No.: US 2026/0182478 P1
(43) Pub. Date: Jun. 25, 2026

(54) Strawberry Plant Named 'CBC024'

(50) ;

(71) Applicant: Kyle M. VandenLangenberg, Tracy, CA (US)

(72) Inventor: Kyle M. VandenLangenberg, Tracy, CA (US)

(73) Assignee: California Berry Cultivars, LLC, French Camp, CA (US)

(21) Appl. No.: 19/420,783

(22) Filed: Dec. 16, 2025

Related U.S. Application Data
(60) Provisional application No. 63/738,159, filed on Dec. 23, 2024.

(51) Int. Cl. A01H 6/74; A01H 5/08

(52) U.S. Cl. CPC A01H 6/7409; A01H 5/08
USPC PLT/208

(57) ABSTRACT

A new and distinct cultivar of strawberry plant (Fragaria x ananassa) named ‘CBC024’ is presented here. This new day-neutral strawberry cultivar is characterized by moderate vigor plants which produce high yields of large, firm, flavorful fruit throughout the growing season. In addition, plants of ‘CBC024’ are resistant to Fusarium oxysporum f. sp fragariae.

### LATIN NAME OF THE GENUS AND SPECIES OF THE PLANT CLAIMED

Botanical classification: Fragaria x ananassa.

### VARIETY DENOMINATION

The new strawberry variety denomination is ‘CBC024’.

### BACKGROUND OF THE INVENTION

This invention relates to a new and distinctive day-neutral type cultivar designated as ‘CBC024’. The new cultivar was the result of a controlled cross performed in 2018 in Oxnard, Calif. between the unpatented selection ‘115.119-101’ and the unpatented selection ‘115.133-101’. ‘CBC024’ was first fruited at an experimental orchard near French Camp, California in 2019, where it was selected, originally designated ‘118.147-107’, and propagated asexually by runners. Following selection and during testing, the plant of this selection was later designated ‘CBC024’. Asexual propagules from this original source have been tested in Watsonville, California, in Oxnard, California, and to a limited extent in grower fields starting in 2023. The cultivar is stable and reproduces true to type in successive generations of asexual reproduction.

### COMPARISON WITH PARENTS

The parent ‘115.119-101’ is a short-day selection chosen for its high yield, firm fruit, and good appearance. Plants of ‘115.119-101’ are larger than ‘CBC024’. The parent ‘115.133-101’ was chosen for its excellent flavor, good appearance, and day-neutral flowering habit. Plants of ‘115.133-101’ are similar in size to ‘CBC024’.

### SUMMARY OF THE INVENTION

‘CBC024’ is a day-neutral cultivar that produces fruit over an extended period when treated appropriately in arid, subtropical climates. When treated with appropriate planting regimes, ‘CBC024’ has larger fruit and produces individual plant yields greater than that of either parent. The following traits have been repeatedly observed over multiple testing years and locations and are determined to be unique characteristics of ‘CBC024’. These characteristics in combination distinguish ‘CBC024’ as a new and distinct Strawberry plant:
a. Medium to large sized plant.
b. Day-neutral flowering habit.
c. Medium glossy red fruit, round to conic shaped.
d. Fruit is firm throughout the growing season.
e. Fruit postharvest longevity is good.
f. Excellent disease resistance.

The cultivar ‘CBC024’, when planted under appropriate conditions, is a continuously flowering day-neutral type with exceptionally high total yields. Fruit quality is good with excellent flavor. In addition to its yield performance, ‘CBC024’ is resistant to Fusarium oxysporum f. sp fragariae, the causal agent of Fusarium wilt in strawberry.

### BRIEF DESCRIPTION OF THE FIGURES

The accompanying color photographs, identified as FIGS. 1 through 5, show the appearance of typical specimens of the new strawberry cultivar designated ‘CBC024’. These Figures depict the colors, as nearly true as can be achieved with conventional photography.

FIG. 1 shows a typical leaf and petiole structure.

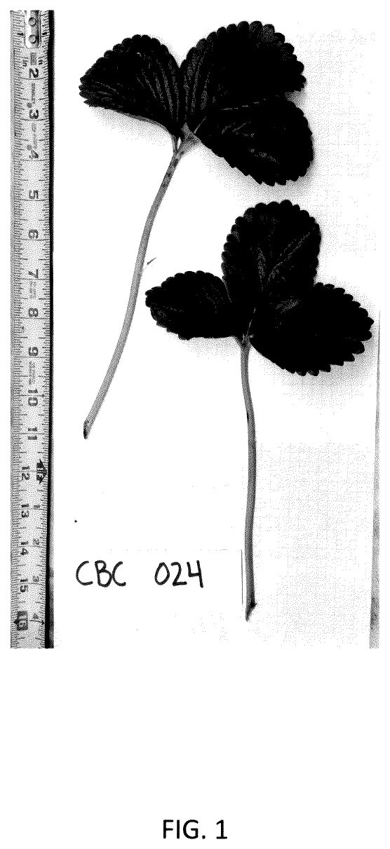

FIG. 2 shows a typical plant and fruit early season.

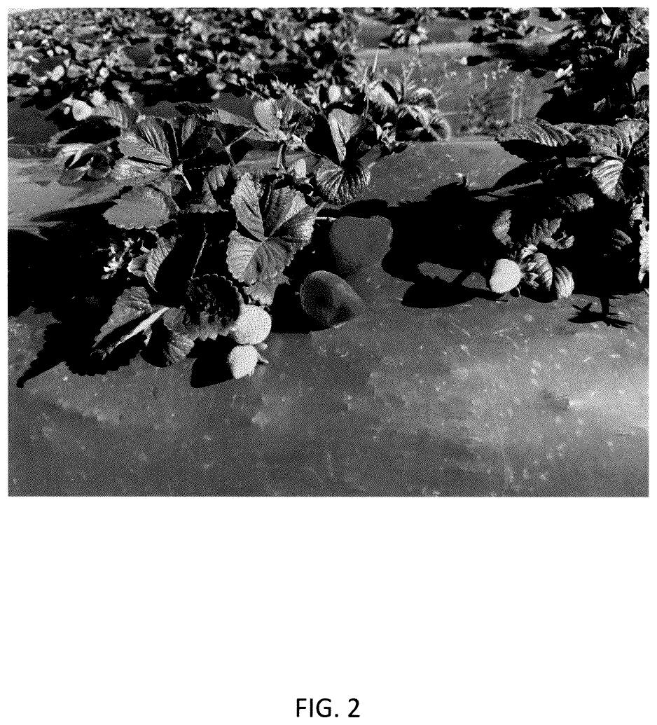

FIG. 3 shows typical fruit in a box.

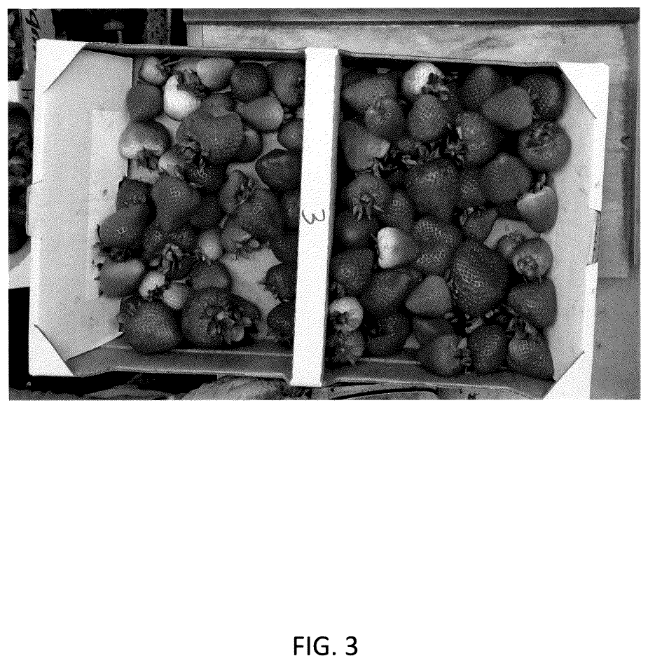

FIG. 4 shows a cross-section of representative fruit mid-season.

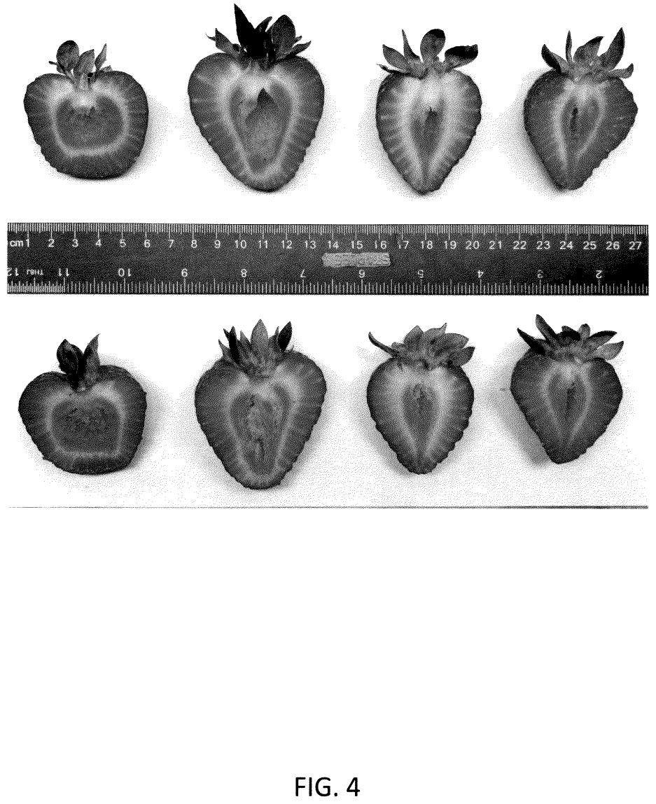

FIG. 5 shows a typical plot in the field of plants and fruit.

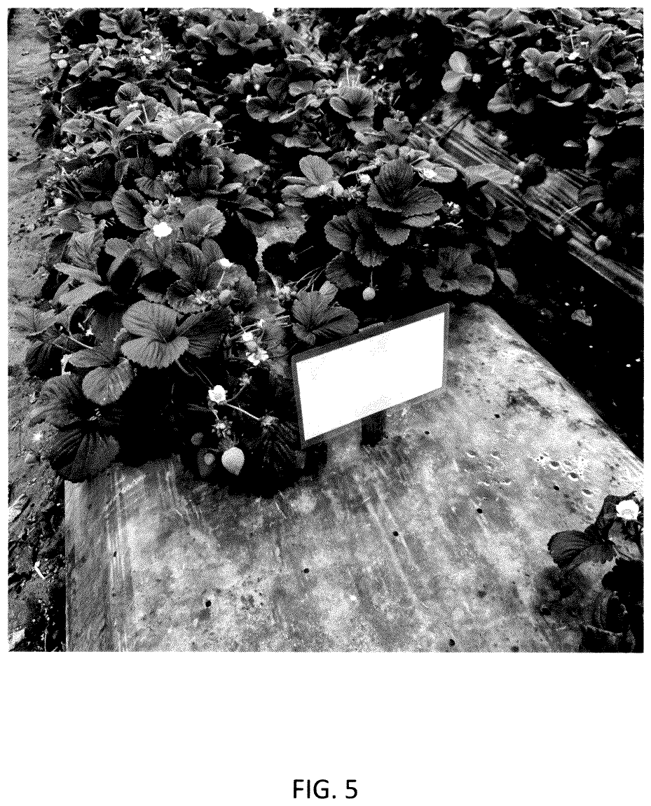

### DETAILED BOTANICAL DESCRIPTION OF THE PLANT

‘CBC024’ is a new and distinct day-neutral cultivar of strawberry, genus, and species Fragaria x ananassa. Plants of ‘CBC024’ bloom continuously in higher elevation plantings as well as the heat of low elevation nursery locations such as French Camp, CA, consistent with other day-neutral varieties. The plants of the new cultivar have a moderate vigor.

‘CBC024’ exhibits several characteristics which are improvements over one or both of its parent varieties, and other cultivars known to the Inventors. These characteristics are compared with other cultivars in the following discussion and tables.

The fruit size of ‘CBC024’ is large sized, larger than its parents. The fruit size of ‘CBC024’ is still within an ideal range since it is not too large to be damaged in shipping containers, but not too small to allow for efficient picking and packing. Storability of the fruit has not been measured but in informal observations the fruit retains good physical characteristics and appearance for several days after harvest at room temperature and up to at least a week in common refrigerator conditions of roughly 3° C.

The data set for ‘CBC024’ set forth below was collected in May 2025 from plants grown at a test plot in Watsonville, CA. Yield, size, firmness data is either averaged over the entire 2024-2025 growing season or presented as monthly totals at the same location. Description data was collected on five-month-old plants.

The observed characteristics of ‘CBC024’ are set forth in Table 1, 2, 3, and 4 below. Color terminology, where noted, is in accordance with the Munsell Colorchart. The observed characteristics of ‘CBC024’ are compared to the closest cultivar known to the Inventor, in Table 2, 3, and 4. Table 5 sets forth the test results of DNA fingerprinting using eight SSR markers.

TABLE 1
Detailed Description of Characteristics of ‘CBC024’

| | |
|---|---|
| SPECIFICATION: | |
| Genus/Species | Fragaria X ananassa |
| Market Name | Strawberry |
| PARENTS: | |
| Female | ‘115.133-101’ |
| Male | ‘115.119-101’ |
| PLANT: | |
| Type | Day-neutral |
| Growth Habit | Upright |
| Foliage density | Moderate |
| Vigor | Moderate |
| Height | Average 24 cm; range 21-27 cm |
| Width | Average 48.6 cm; range 46-51 cm |
| Disease tolerance | Test plots have shown tolerance to |
| | Fusarium oxysporum f. sp fragariae |
| LEAF: | |
| Width | 16 cm |
| Color | Adaxial surface: 5 GY 4/3 |
| | Abaxial surface: 7.5 GY 5/7 |
| Pubescence | Slight |
| Glossiness | Low |
| Variegation | Not present |
| TERMINAL LEAFLET | |
| Length | Average 76 mm; range 68-88 mm |
| Width | Average 65 mm; range 53-81 mm |
| Ratio (length/width) | 1.17 |
| Margins | Crenate |
| Leaflet shape | Obovate |
| Base shape | Cuneate |
| Cross-section shape | Concave |
| PETIOLE | |
| Pubescence | High Density |
| Petiole color | 5 GY 8/8 |
| Petiole length | Average 14 cm; range 13-16 cm |
| Petiole diameter | Average 4 mm; range 3-5 mm |
| STOLON | |
| Anthocyanin coloration | Present, medium |
| Pubescence | Slight |
| Diameter | Average 3 mm; range 3-3 mm |
| STIPULE | |
| Anthocyanin coloration | Present, dark |
| Length | Average 24 mm; range 19-28 mm |
| Width | Average 16 mm; range 14-18 mm |
| INFLORESCENCE: | |
| Flowering | Early to Normal |
| Position | At or above canopy |
| Petal arrangement | Moderate overlap |
| Calyx size | Normal to large |
| Number of blooms | 12 |
| Length | 2.5 cm |
| Flower diameter | Average 27 mm; range 23-32 mm |
| Petal number | Average 7; range 6-8 |
| Petal length | Average 11 mm; range 10-13 mm |
| Petal width | Average 9.7 mm; range 8-12 mm |
| Ratio (length/width) | 1.13 |
| Petal color | N 9.5/0 |
| Calyx color | Adaxial surface: 7.5 GY 5/7 |
| | Abaxial surface: 7.5 GY 8/7 |
| Stamens length | Average 22 mm; range 21-24 mm |
| PEDICEL | |
| Pubescence | Slight |
| Length | Average 173 mm; range 159-245 mm |
| Width | Average 5.1 mm; range 4-6 mm |
| Ratio (length/width) | 33.9 |
| Color | 5 GY 8/8 |
| FRUIT: | |
| Bearing | Day-neutral |
| Individual fruit weight | Seasonal average: 35.5 g |
| Shape | Cordate to broad conic |
| Achenes | Flush to slightly embedded |
| Glossiness | High gloss |
| External color evenness | Even external color, excellent |
| External color | 7.5 R 4/11 |
| Internal color | 10 R 8/6 |
| Band at top without | Narrow |
| achenes | |
| Fruit Yield | Grams per plant: 2902 g |
| Firmness | 0.51 kg/force |
| Achene color | 10 Y 7/9 |

TABLE 2
Marketable yield of ‘CBC024’ compared to other cultivars known
to the Inventor in Watsonville, CA during the 2024-2025 season:
‘Cabrillo’, U.S. Plant Pat. No. 27,830 and
‘Monterey’, U.S. Plant Pat. No. 19,767.

| | Marketable yield (g/plant) | | | | | |
|---|---|---|---|---|---|---|
| Cultivar | Apr. | May | Jun. | Jul. | Aug. | Total |
| ‘CBC024’ | 496 | 610 | 625 | 764 | 407 | 2902 |
| ‘Cabrillo’ | 186 | 415 | 717 | 800 | 218 | 2336 |
| ‘Monterey’ | 195 | 485 | 648 | 600 | 178 | 2105 |

TABLE 3
Size of ‘CBC024’ compared to other cultivars known
to the Inventor in Watsonville, CA during the 2024-2025season.
Fruit size (g/fruit)

| Cultivar | Apr. | May | Jun. | Jul. | Aug. | Average |
|---|---|---|---|---|---|---|
| ‘CBC024’ | 48.4 | 40.4 | 34.6 | 30.0 | 24.3 | 35.5 |
| ‘Cabrillo’ | 37.0 | 37.9 | 33.5 | 27.9 | 23.6 | 32.0 |
| ‘Monterey’ | 41.2 | 39.2 | 30.0 | 23.2 | 19.7 | 30.7 |

TABLE 4
Firmness of ‘CBC024’ compared to other cultivars known
to the inventor in Watsonville, CA during the 2024-2025 season.
Firmness (kg/force)

| Cultivar | May | Jun. | Jul. | Average |
|---|---|---|---|---|
| ‘CBC024’ | 0.52 | 0.51 | 0.51 | 0.51 |
| ‘Cabrillo’ | 0.60 | 0.55 | 0.56 | 0.57 |
| ‘Monterey’ | 0.56 | 0.51 | 0.50 | 0.52 |

TABLE 5
DNA finger printing results of eight SSR markers.

| | Allelic Fingerprint Analysis | | | | | | | |
|---|---|---|---|---|---|---|---|---|
| Cultivar | M1 | M2 | M3 | M4 | M5 | M6 | M7 | M8 |
| ‘CBC024’ | 206, | 259, | 188, | 279, | 156 | 238, | 121, | 213, |
| | 222, | 264, | 190, | 281, | | 242, | 124, | 215, |
| | 224 | 271, | 216, | 289 | | 251, | 130, | 222, |
| | | 275 | 232 | | | 256, | 146 | 228 |
| | | | | | | 257, | | |
| | | | | | | 262 | | |

I claim:

1. A new and distinct strawberry plant named ‘CBC024’ as described and illustrated herein.
